(19) United States
(12) Patent Application Publication
(10) Pub. No.: US 2022/0387207 A1
(43) Pub. Date: Dec. 8, 2022

(54) SHOULDER BRACE AND METHOD

(71) Applicant: William R. Post., Morgantown, WV (US)

(72) Inventor: William R. Post., Morgantown, WV (US)

(21) Appl. No.: 17/338,364

(22) Filed: Jun. 3, 2021

(51) Int. Cl. A61F 5/37; A61F 7/10

(52) U.S. Cl. CPC A61F 5/3738; A61F 7/10; A61F 2007/003

(57) ABSTRACT

A shoulder brace includes a base layer having a first side and a second side, a side support coupled to the first side, and a sling attached to the side support and having a sling strap. A counterforce strap has a first end coupled to the second side of the base layer, and a second end attached to a portion of the sling strap. So configured, the counterforce strap applies a force to the sling strap in a direction toward the second side of the base layer to relieve pressure on a user's neck during use. In addition, a cold therapy device assembly is coupled to the shoulder brace and includes a cold therapy device having first and second portions and at least one connecting member attached to one of the first and second portions and configured to be attached to a portion of the shoulder brace.

### TECHNICAL FIELD

The present disclosure relates to shoulder braces and, more specifically, to a shoulder brace having a counterforce strap accessible by a user when applying and in use.

### BACKGROUND

Many conventional shoulder braces and slings include a narrow strap around a back area of a user and/or a strap across both a mid-back area and an opposite underarm of the user, both of which can be very uncomfortable for the user. In addition, conventional shoulder braces are often very difficult for the user to apply on her own without assistance from another person. This is due at least in part to the location of some straps of the conventional shoulder braces that are too difficult for the user to independently access when using the shoulder brace. Moreover, many conventional shoulder braces are also too difficult to apply in an operating room immediately post-operation due to these same deficiencies. Still further, some current shoulder braces include a handgrip in the sling, and the handgrip is attached to both sides of the sling, making it difficult for the user to slide her arm in and out of the sling. Braces that are not correctly applied can have detrimental effects on patient comfort and healing.

### SUMMARY

In accordance with a first exemplary aspect, a shoulder brace comprises a base layer having a first side and a second side, a side support coupled to the first side, and a sling attached to the side support and having a sling strap. A counterforce strap has a first end coupled to the second side of the base layer and a second end attached to a portion of the sling strap. So configured, the counterforce strap applies a force to the sling strap in a direction toward the second side of the base layer to relieve pressure on the neck from the sling strap.

In accordance with another exemplary aspect, a method of supporting an arm of a user comprises providing a base layer of a shoulder brace, the base layer configured to be disposed around a waist of a user, and coupling a side support to a first side of the base layer. The method also includes attaching a sling to the side support, the sling for receiving an arm of the user and having a sling strap configured to be disposed over a shoulder of the user to support the weight of the sling. The method further includes securing a first end of a counterforce strap to a second side of the base layer, and securing a second end of the counterforce strap to a portion of the sling strap, applying a force to the sling strap in a direction toward the second side of the base layer.

In accordance with another exemplary aspect, a cold therapy device assembly for use with a shoulder brace comprises a cold therapy device having a first portion and a second portion, at least one connecting member coupled to the first portion of the cold therapy device, and at least one connecting member coupled to the second portion of the cold therapy device. So configured, the at least one connecting member coupled to the first portion of the cold therapy device is configured to be attached to a portion of a sling or a shoulder brace, and the at least one connecting member coupled to the second portion of the cold therapy device is configured to be attached to another portion of the sling or the shoulder brace.

In accordance with yet another exemplary aspect, a method of applying a cold therapy device assembly to a shoulder using one of a sling or a shoulder brace comprises providing a cold therapy device adapted to be disposed on a shoulder of a user. The method also includes attaching a first portion of the cold therapy device to a portion of a sling or a shoulder brace by at least one connecting member and attaching a second portion of the cold therapy device to another portion of the sling or the shoulder brace by at least one other connecting member.

In one form, the shoulder brace may comprise a hand grip attached to only one side of the sling.

In another form, the base layer may be configured to be disposed around a waist of the user. In addition, the base layer may further comprise additional padding for lumbar support disposed on the first side of the base layer.

In yet another form, the side support may be coupled to the first side of the base layer by a hook and loop mechanism.

In still another form, the first side of the base layer may be configured to be disposed near a first hip of the user. In addition, the second side of the base layer may be configured to be disposed near a second hip of the user.

In another form, the first side of the base layer may be disposed opposite to the second side of the base layer.

In yet another form, the sling strap may include a first end coupled to a first portion of the sling, a second end coupled to the second portion of the sling, and a middle area disposed between the first and second ends. The middle area may be configured to extend across a shoulder of the user.

In another form, the first portion of the sling may be a front portion of the sling and the second portion of the sling may be a rear portion of the sling.

In yet another form, the second end of the counterforce strap may be coupled to the middle portion of the sling strap. In addition, a section of the middle portion may be configured to be disposed on a chest of the user.

In still another form, the sling strap may include a ring. In addition, the second end of the counterforce strap may attach to the ring to relieve weight of the sling strap on a neck of the user.

In another form, the sling may include a body having a front portion, a rear portion, and an inside surface extending from the front portion to the rear portion. The sling may also include a hand grip attached to the inside surface on only one side of the body near the front portion, allowing a hand of a user to be easily inserted into the body of the sling to contact the hand grip.

In yet another form, the side support may be adjustably coupled to the base layer to accommodate various placements of the sling.

In another form, the shoulder brace may further comprise a support strap having a first end attached to the second side of the base layer and a second end removably attached to the side support disposed at the first side of the base layer, opposite to the second side of the base layer, to support the base layer.

In another form, the shoulder brace may comprise extra padding disposed within the base layer for user comfort.

In yet another form, the shoulder brace may include a cold therapy device assembly attached to a portion of the sling strap or sling.

In another form, the cold therapy device assembly may include a cold therapy device having a first portion and a second portion, at least one connecting member coupled to the first portion of the cold therapy device, and at least one connecting member coupled to the second portion of the cold therapy device. The at least one connecting member coupled to the first portion of the cold therapy device may be attached to a front portion of the sling strap, and the at least one connecting member coupled to the second portion of the cold therapy device may be attached to a rear portion of the sling strap.

In another form, securing a second end of the counterforce strap to a portion of the sling strap may comprise securing a second end of the counterforce strap to one or more of: (1) a portion of the sling strap configured to be disposed on a front area of the user; (2) a middle portion of the sling strap; and (3) a ring disposed on the sling strap.

In yet another form, the method may further comprise attaching a first end of a support strap to the second side of the base layer and removably attaching a second end of the support strap to the side support disposed at the first side of the base layer, opposite to the second side of the base layer, to support the base layer.

In yet another form, the method may further comprise coupling a first end of the sling strap to a first portion of the sling configured to be disposed near a front area of the user.

In another form, the method may further comprise disposing additional padding for lumbar support on or in the base layer.

In yet another form, the method may further comprise attaching a hand grip to an inside surface on one side of a body of the sling, allowing a hand of a user to be easily inserted into the body of the sling to contact the hand grip.

In yet another form, the at least one connecting member coupled to the first portion of the cold therapy device may be at least one connecting strap and the at least one connecting member coupled to the second portion of the cold therapy device may be at least one connecting strap, wherein each of the at least one connecting straps includes a set of connecting straps.

In another form, the cold therapy device assembly may comprise a first set of connecting straps having at least one connecting strap configured to be attached to a front portion of a sling strap of the shoulder brace and a second set of connecting straps having at least one connecting strap configured to be attached to a rear portion of the sling strap of the shoulder brace.

In still another form, the at least one connecting member coupled to the first portion of the cold therapy device may be a first set of connecting straps having a first connecting strap and a second connecting strap. The first connecting strap may have a first portion attached to a front portion of the cold therapy device, and a second portion configured to be disposed through a ring in the front portion of the sling strap. In addition, the second connecting strap may have a first portion coupled to the front portion of the cold therapy device and a second portion attached to a side portion of the cold therapy device. The side portion may be disposed on an upper arm area of a user.

In yet another form, the at least one connecting member coupled to the second portion of the cold therapy device may comprise a second set of connecting straps having a first connecting strap and a second connecting strap. The first connecting strap may be disposed over a rear portion of the sling strap and onto a rear portion of the cold therapy device, and the second connecting strap may have a first portion attached to the rear portion of the cold therapy device and a second portion attached to a side portion of the cold therapy device.

In another form, the cold therapy device may include one of a gel pad, an ice bag, a contoured device that circulates water to maintain consistent cryotherapy, or another pad that contours to the body, such as the shoulder.

Any one or more of these aspects may be considered separately and/or combined with each other in any functionally appropriate manner. In addition, any one or more of these aspects may further include and/or be implemented in any one or more of the optional exemplary arrangements and/or features described hereinafter. These and other aspects, arrangements, features, and/or technical effects will become apparent upon detailed inspection of the figures and the following description.

### BRIEF DESCRIPTION OF THE DRAWINGS

The features of this disclosure which are believed to be novel are set forth with particularity in the appended claims. The present disclosure may be best understood by reference to the following description taken in conjunction with the accompanying drawings, in which like reference numerals identify like elements in the several figures, and in which:

FIG. 1 is a front view of a shoulder brace according to the present disclosure;

FIG. 2 is a perspective view of a base layer and a sling of the shoulder brace of FIG. 1;

FIG. 3 is rear view of the base layer and the sling of the shoulder brace of FIG. 1;

FIG. 4 is a front, perspective view of the base layer and the sling of the shoulder brace of the present disclosure;

FIG. 5 is a side, perspective view of a portion of the shoulder brace of the present disclosure, depicting a support strap of the shoulder brace;

FIG. 6 is another side, perspective view of the shoulder brace of the present disclosure, depicting a counterforce strap of the shoulder brace;

FIG. 7 is a side view of the shoulder brace of the present disclosure;

FIG. 8 is a top, perspective view of the sling of the shoulder brace of the present disclosure;

FIG. 9 is side, perspective view of the sling of the shoulder brace of the present disclosure;

FIG. 10 is another side, perspective view of the sling of the shoulder brace;

FIG. 11 is a rear view of the shoulder brace of FIG. 1;

FIG. 12 is a front view of a shoulder brace and cold therapy device according to another aspect of the present disclosure; and

FIG. 13 is a rear view of the shoulder brace and cold therapy device of FIG. 12.

### DETAILED DESCRIPTION

A shoulder brace including a base layer and a counterforce strap that relieves pressure of a sling strap against a neck of a user is disclosed. Generally, the counterforce strap is disposed in an area a user can easily access and connect, which is unlike many conventional braces, in which straps are difficult to access behind the back and/or disposed under an arm opposite to the arm the sling receives, pulling up into the armpit and adding to discomfort for the user. Application of the base layer of the shoulder brace of the present disclosure allows a sling to be attached in a position of comfort. In addition, straps secure the base layer to the user's waist, and the counterforce strap extends from an area of the shoulder brace opposite to the location of the sling. So configured, the user is able to easily access the straps and independently operate and use the shoulder brace of the present disclosure.

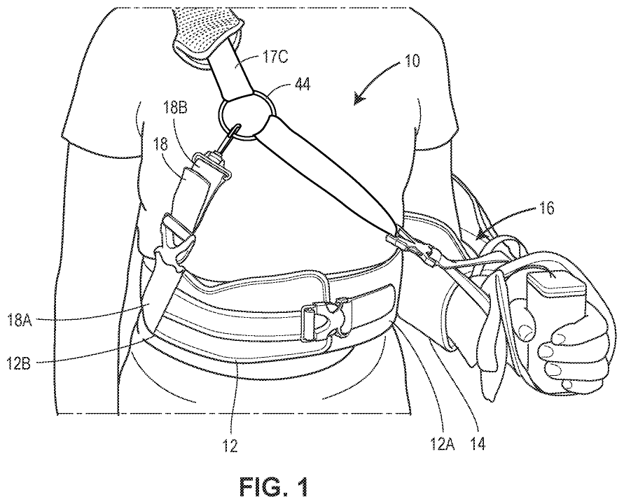

Referring now to FIG. 1, a shoulder brace 10 of the present disclosure is depicted. The shoulder brace 10 includes a base layer 12 having a first side 12A and a second side 12B disposed opposite to the first side 12A. In this example, and as depicted, the shoulder brace 10 is disposed around a waist of a user, e.g., such as a patient. Specifically, the first side 12A of the base layer 12 is disposed near a first hip of the user, and the second side 12B is disposed near a second hip of the user.

In addition, and still referring to FIG. 1, the shoulder brace 10 also includes a side support 14 coupled to the first side 12A of the base layer 12. A sling 16 is attached to the side support 14 and has a sling strap 17 configured to be disposed over a shoulder of the user. The shoulder brace 10 further includes a counterforce strap 18 having a first end 18A coupled to the second side 12B of the base layer 12 and a second end 18B removably attached to a portion of the sling strap 17, as depicted in FIG. 1. So configured, the counterforce strap 18 applies a force to the sling strap 17 in a direction toward the second side 12B of the base layer 12, thereby removing pressure on a neck area of the user when using the shoulder brace 10, as explained more below.

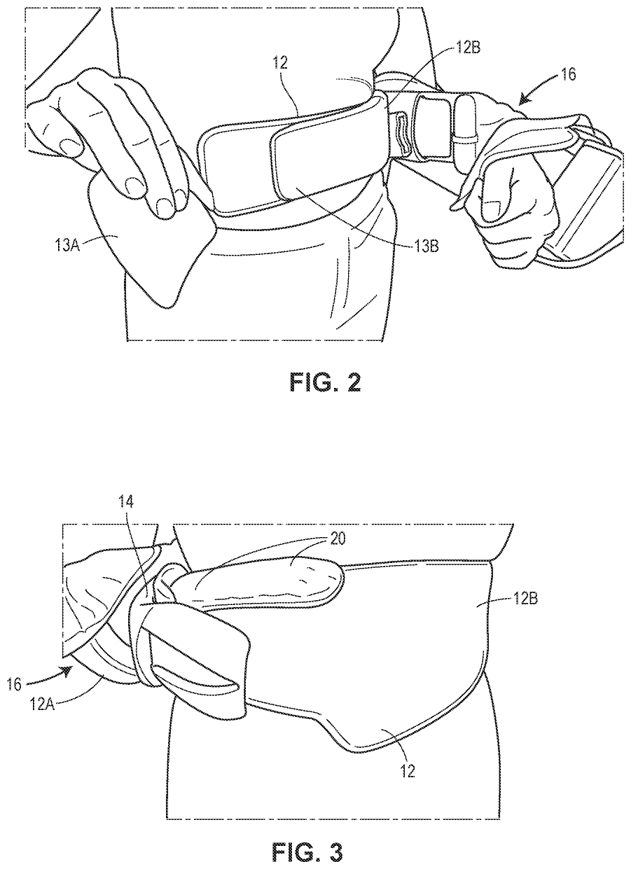

Referring now to FIG. 2, the base layer 12 is depicted disposed around the waist of the user. The base layer 12 also includes a first end 13A and a second end 13B. During use, the base layer 12 is first wrapped around the user's waist, and then the first end 13A is secured to the second end 13B of the base layer 12 using a hook and loop configuration, such as commonly known Velcro. Specifically, the second end 13B may have a loop configuration attached thereto, and the first end 13A may have a hook configuration attached thereto, such that when the first end 13A is disposed over and in contact with the second end 13B of the base layer 12, the hook configuration of the first end 13A attaches to the loop configuration of the second end 13B to secure the first and second ends 13A, 13B of the base layer 12 together and around the user's waist. Of course, it will be appreciated that various other attachment mechanisms different from a Velcro configuration, but operating to effectively secure the first and second ends 13A, 13B together, may alternatively be used and still fall within the scope of the present disclosure.

Referring now to FIG. 3, a rear view of the base layer 12 of the shoulder brace 10 is depicted. The base layer 12 includes additional padding 20, such as for lumbar support, disposed one or more of within the base layer 12, such as a rear area of the base layer, and/or on or near the first side 12A of the base layer 12. In addition, and in one example, the side support 14 is coupled to the base layer 12 by a hook and loop configuration, again such as Velcro. It will again be understood that various other attachment mechanisms operating in the same manner may alternatively be used to couple the side support 14 to the base layer 12 and still fall within the scope of the present disclosure.

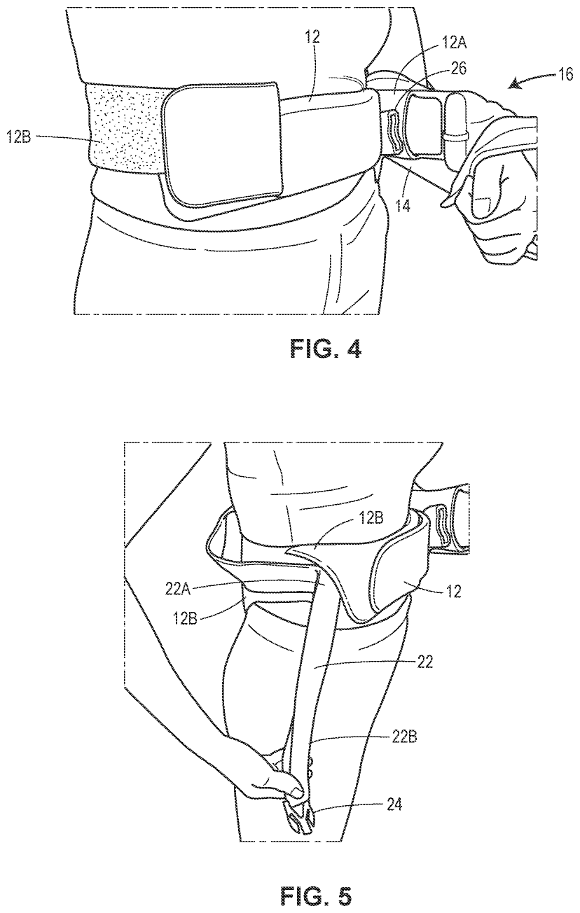

The side support 14 is adjustable, in that it may be placed at various different positions and/or areas of the attachment area of the base layer 12, for example, to account for various heights of users and varied positions of support for the arm being supported, such as the position depicted in FIG. 4. In this position, the side support 14 is immediately adjacent to the user's elbow, allowing the sling 16 that receives the elbow and arm of the user, to be easily attached to the side support 14. In addition, the side support 14 may be angled or take the form of various shapes and still fall within the scope of the disclosure.

As also depicted in FIG. 3, the base layer 12 includes a width much greater than a width of a strap that is often disposed around a waist of the user in conventional shoulder braces, for example. In one example, the width of the base layer 12 is at least 3 times greater than a width of the sling strap 17, for example, and may be greater in other examples. The larger width of the base layer 12 enables padding to be disposed therein and is more comfortable for a user during use.

Referring now to FIG. 5, the shoulder brace 10 further includes a support strap 22 having a first end 22A attached to the second side 12B of the base layer 12, and a second end 22B disposed opposite to the first end 22A. The second end 22B is removably attached to the side support 14 disposed at the first side 12A of the base layer 12, opposite to the second side 12B of the base layer 12, to support the base layer 12 during use of the shoulder brace 10, for example. In one example, the second end 22B may include a clip portion 24 that is removably inserted into a clip receiving portion 26 disposed on the side support 14, as depicted in FIG. 4. By having the first end 22A of the support strap 22 attached to the second side 12B of the base layer 12, the support strap 22 is easily accessible by the user of the shoulder brace 10. In addition, the clip portion 24 is easily insertable into the clip receiving portion 26, which is also easily accessible by the user due to its placement on the side support 14 adjacent to the first side 12A of the base layer 12, for example.

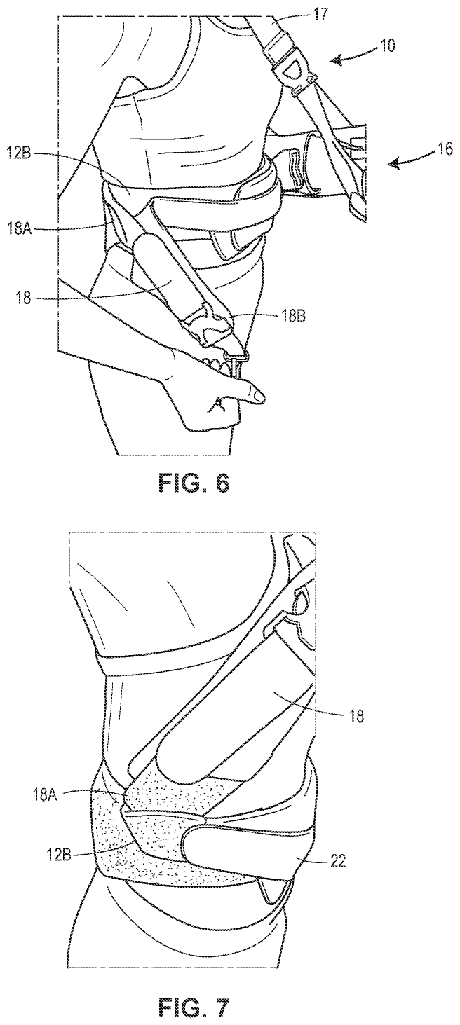

Referring now to FIGS. 6 and 7, the counterforce strap 18 of the shoulder brace 10 is further depicted. Specifically, FIG. 6 depicts the counterforce strap 18 in an unapplied state, and FIG. 7 depicts in the counterforce strap 18 in an applied state in which the counterforce strap 18 is coupled to the sling strap 17 of the sling 16, as previously explained. When in the applied state, the counterforce strap 18 relieves pressure from the sling strap 17 on the neck of the user, providing a more comfortable shoulder brace. More specifically, the second end 18B of the counterforce strap 18 is coupled to a portion of the sling strap 17 that is disposed on front chest of the user, as depicted in FIG. 1.

The location of the first end 18A of the counterforce strap 18 on the second side 12B of the base layer 12 again makes the counterforce strap 18, and in particular the second end 18B of the counterforce strap 18, easily accessible to the user when operating the shoulder brace 10. Further, when the counterforce strap 18 is in the applied state, such as depicted in FIG. 7, the counterforce strap 18 rests comfortably below an armpit of the user opposite to the armpit of the arm disposed within the sling 16. As a result, no strap of the shoulder brace 10 is disposed under an arm or across the armpit of the user, further increasing the comfort level of the shoulder brace 10.

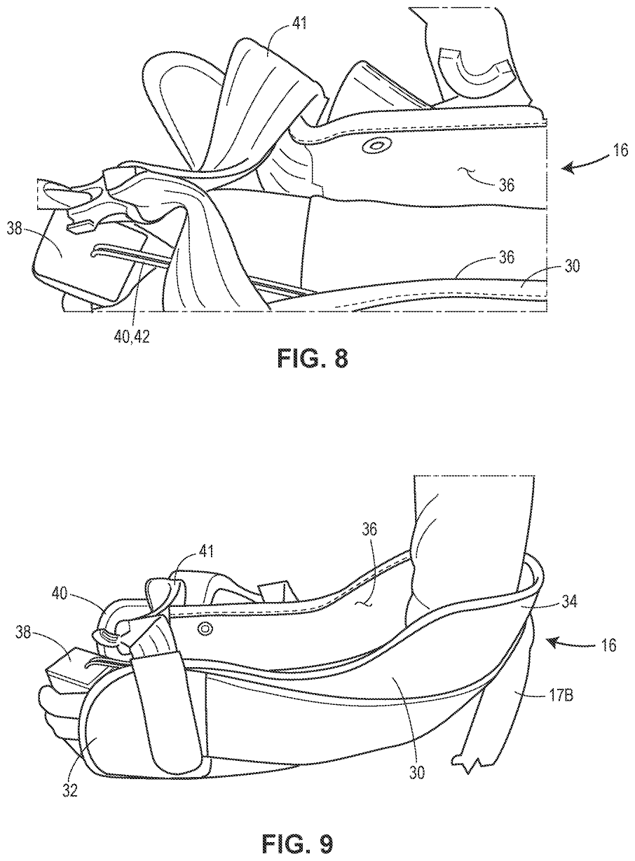

Referring now to FIGS. 8 and 9, the sling 16 includes a body 30 having a front portion 32, a rear portion 34, and at least one inside surface 36 extending from the front portion 32 to the rear portion 34. In FIG. 8, two inside surfaces 36 are depicted, one extending from the front portion 32 to the rear portion 34 along one side of the body 30, and the other extending from the front portion 32 to the rear portion 34 along the other side of the body 30 of the sling 16. A hand grip 38 is attached to the inside surface 36 disposed on one side of the body 30 near the front portion 32 of the sling 16. This allows a hand of the user to be easily inserted into the body 30 of the sling 16 to contact and use the hand grip 38. In one example, the hand grip 38 is attached to the inside surface 36 via an attachment member 40, such as an attachment strap 42, that is secured to the inside 36 on one side of the body 30 of the sling 16, as depicted in FIG. 8.

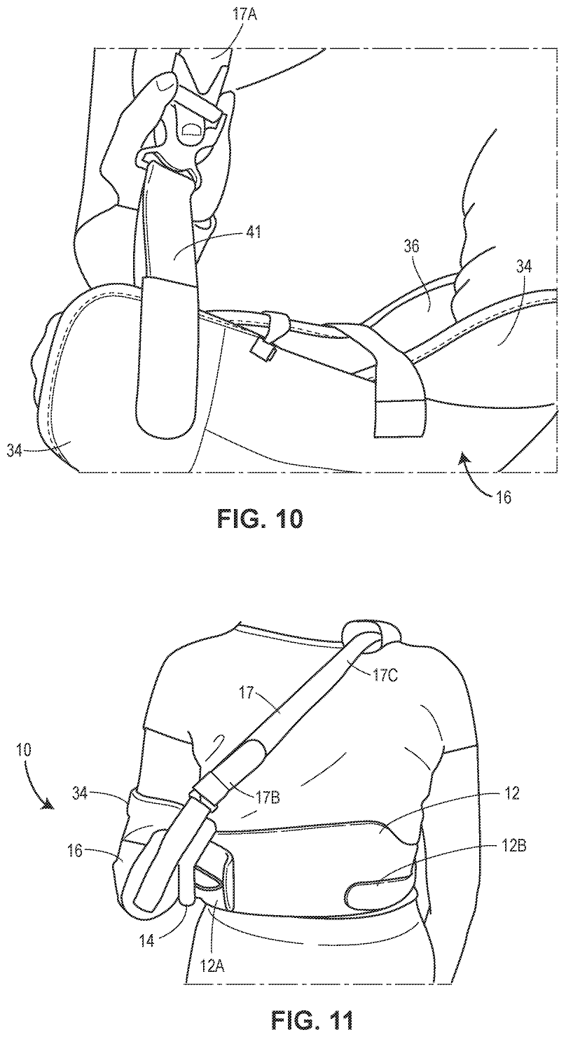

As depicted in FIG. 10, the sling strap 17 of the sling 16 includes a first end 17A ultimately coupled to a first portion, such as the front portion 32, of the sling 16. Specifically, in this example, the first end 17A of the sling strap 17 is coupled to a connecting member 41 disposed near the front portion 32 of the sling 16. The connecting member 41 may include a strap having a receiving member into which a clip of the first end 17A of the sling strap 17 is inserted. Of course, it will be understood that many other attachment mechanisms may alternatively and/or additionally be used to couple the first end 17A of the sling strap 17 to the sling 16 and still fall within the scope of the present disclosure.

Referring now to FIG. 11, the sling strap 17 also includes a second end 17B (see also FIG. 9) coupled to a second portion, such as the rear portion 34, of the sling 16. The second end 17B of the sling 16 may be coupled to the sling 16 using any known manner, including a clip or other attachment mechanism. As further depicted in FIG. 11, the middle portion 17C of the sling strap 17 is also configured to extend across and along a back portion of the user and over a shoulder of the user opposite to the shoulder of the affected arm.

Referring back to FIG. 1, the middle portion 17C of the sling strap 17 also extends over the shoulder of the user opposite to the shoulder of the affected arm and across a front portion of the user's chest, as depicted in FIG. 1. In this example, a middle portion 17C of the sling strap 17 configured to extend across the user's chest may also include a ring 44. The second end 18B of the counterforce strap 18 attaches to the ring 44 to relieve weight of the sling strap 17 on the neck of the user. While the ring 44 is depicted, it will be understood that various other types of attachment mechanisms may additionally and/or alternatively be used to secure to the second end 18B of the counterforce strap 18 to the ring 42 and still fall within the scope of the present disclosure.

In any case, if the first end 17A of the sling strap 17 is detached from the sling 16 during application of the shoulder brace 10, the user is able to easily access the first end 17A of the sling strap 17, place the middle portion 17C of the sling strap 17 over her shoulder, and then couple the first end 17A of the sling strap 17 to the front end 32 of the sling 16, as depicted in FIG. 10. In another example, the ends 17A and 17B of the sling strap 17 may always be kept in a position in which both ends 17A, 17B are coupled to the sling 16, allowing the user to easily place the sling strap 17 over her head and shoulder when applying the shoulder brace, without having to couple the first end 17A of the sling strap 17 to the sling 16 as well during the application.

In view of the foregoing, it will be understood that a method of supporting the arm of the user may include using the shoulder brace 10 of the present disclosure. Specifically, the method may include providing the base layer 12 of the shoulder brace 10, which is configured to be disposed around the waist of the user. The method may also include coupling the side support 14 to the first side 12A of the base layer 12 and attaching the sling 16 to the side support 14, the sling 16 including the sling strap 17. The method may still also include securing the first end 18A of the counterforce strap 18 to the second side 12B of the base layer 12 and the second end 18B of the counterforce strap 18 to a portion of the sling strap 17, thereby applying a force to the sling strap 17 in a direction toward the second side 12B of the base layer 12.

In one example, securing the second end 18B of the counterforce strap 18 to a portion of the sling strap 17 includes securing the second end 18B of the counterforce strap 18 to one or more of: (1) a portion of the sling strap 17 configured to be disposed on a front area of the user; (2) the middle portion 17C of the sling strap 17; or (3) the ring 42 disposed on the sling strap 17.

In other examples, the method may further include attaching the first end 22A of the support strap 22 to the second side 12B of the base layer 12 and removably attaching the second end 22B of the support strap 22 to the side support 14 disposed at the first side 12A of the base layer 12 to support the base layer 12. In addition, the method may further include coupling the first end 17A of the sling strap to the first portion 32 of the sling 16 configured to be disposed near a front area of the user. Further, the method may also include disposing additional padding for lumbar support on the first side 12A of the base layer 12 and/or within any area in or on the base layer 12. Lastly, the method may still further include attaching the hand grip 38 to the inside surface 36 of the body 30 of the sling 16, allowing the hand of the user to be easily inserted into the body 30 of the sling 16 to contact and use the hand grip 38.

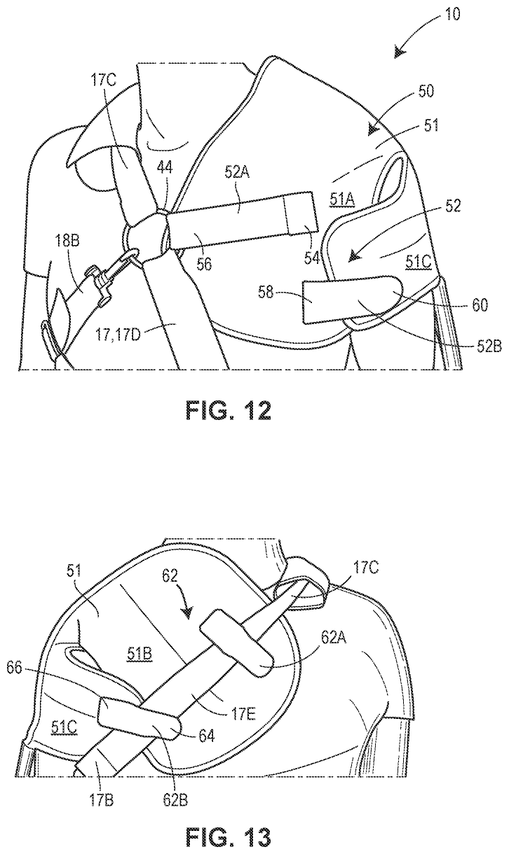

Referring now to FIGS. 12 and 13, the shoulder brace 10 of FIGS. 1-11 and the foregoing description is depicted with a cold therapy device assembly 50 attached thereto. Specifically, the shoulder brace 10 of FIGS. 12 and 13 includes all of the same features describe above relative to FIGS. 1-11, for example, except for the cold therapy device assembly 50. Thus, for the sake of clarity and brevity, parts of the shoulder brace 10 described above use the same reference numbers, but are not described again.

As depicted in FIG. 12, the cold therapy device assembly 50 includes a cold therapy device 51 that is attached to the sling strap 17 of the shoulder brace 10 of the present disclosure. Specifically, in this example, the sling strap 17 further includes a front portion 17D and a rear portion 17E (FIG. 13). The cold therapy device assembly 50 also includes a first set of connecting members 52 having at least one connecting member attached to the front portion 17D of the sling strap 17 and a second set of connecting members 62 (FIG. 13) having at least one connecting member attached to the rear portion 17E of the sling strap 17.

More specifically, and in one example, the first set of connecting members 52 may include a first connecting strap 52A and a second connecting strap 52B. The first connecting strap 52A has a first portion 54 attached to a front portion 51A of the cold therapy device 51 and a second portion 56. The second portion 56 is disposed through the ring 44 of the sling strap 17, here the front portion 17D of the sling strap 17, to secure the first connecting strap 52A to the sling strap 17 of the sling. In addition, the second connecting strap 52B likewise has a first portion 58 coupled to the front portion 51A of the cold therapy device 51 adjacent to the first portion 54 of the first connecting strap 52A and a second portion 60. The second portion 60 is attached to a side portion 51C of the cold therapy device 51. In this example, the side portion 51C is disposed on an upper arm area of the user and over the front portion 51A of the cold therapy device 51. However, it will be appreciated that the side portion 51C may alternatively be disposed under the front portion 51A of the cold therapy device 51 and still fall within the scope of the present disclosure.

Further, and in a manner similar to the first set of connecting members 52, the second set of connecting members 62 (FIG. 13) includes a first connecting strap 62A and a second connecting strap 62B. The first connecting strap 62A is disposed over the rear portion 17E of the sling strap 17 and onto a rear portion 51B of the cold therapy device 51 to secure the cold therapy device 50 to the sling strap 17. The second connecting strap 62B has a first portion 64 attached to the rear portion 51B of the cold therapy device 51 and a second portion 66 attached to the side portion 51C of the cold therapy device 51, as depicted in FIG. 13.

As will be appreciated, the cold therapy device 50 may include one of a gel pad, an ice bag, any contoured device that circulates cold water to maintain consistent cryotherapy, or any other pad that contours to the body, such as the shoulder, of the user.

While the cold therapy device 51 is depicted attached to the sling strap 17, it will be understood that the cold therapy device 51 may alternatively be attached to another portion of the sling 16 and/or more generally the shoulder brace 10 and still fall within the scope of the present disclosure. For example, the cold therapy device 50 may be attached to a portion of the body 30 of the sling 16. In addition, the cold therapy device 50 may be attached to a portion of the base layer 12 or any other part of the shoulder brace 10 and still fall within the scope of the present disclosure.

Further, while in one example the first and second sets of connecting members 52, 62 are straps used to secure the cold therapy device 51 to the shoulder brace 10, it will be appreciated that any other attachment mechanism able to operate in the same manner as the first and second connecting members 52, 62 may alternatively and/or additionally be used and still fall within the scope of the present disclosure. For example, instead of the connecting straps, the cold therapy device 51 may be attached to any portion of the shoulder brace 10, such as the sling strap 17, using a ring and clip combination. Said another way, the first and second connecting members 52, 62 may include one or more rings or one or more clips attached to the front and rear portions of the cold therapy device 51, similar to the locations of the sets of straps 52, 62 depicted in FIGS. 12 and 13. So configured, the clips will be attached to corresponding rings by a user to attach the cold therapy device 51 to the shoulder brace 10. In addition, it will be appreciated that any other attachment mechanism operating to attach a portion of the cold therapy device 51 to the sling 16 or shoulder brace 10 may be used and still fall within the scope of the present disclosure.

More generally, while the cold therapy device 51 is depicted attached to the shoulder brace 10 of FIGS. 1-11, the cold therapy device 50 may alternatively be attached to any other shoulder brace or sling and still fall within the scope of the present disclosure. Specifically, the sets of connecting members 52, 62 or any other attachment mechanism as described above of the cold therapy device 50 may be attached to a portion of a shoulder brace or sling and enjoy the same benefits of the cold therapy device 50 attached to the shoulder brace 10 of FIGS. 1-11, as described more described below.

In view of the foregoing, it will be appreciated that the shoulder brace 10 and methods of the present disclosure include several advantages. For example, the shoulder brace 10 and methods provide the comfortable and secure base layer 12 around the waist of the user with the advantage of extra padding and lumbar support. In addition, users are able to independently apply various parts of the shoulder brace 10 without assistance from another person. For example, once the base layer 12 is applied, the user is able to place the sling strap 17 over her head. The user may then use their opposite hand to easily reach both the support strap 22 and the counterforce strap 18, which have ends on the second side 12B of the base layer at the opposite hip of the sling 16 near the healthy arm and not around the user's back, for example. Moreover, there is no strap across the mid-section of the back of the user, and there is no strap across the opposite armpit of the user, due at least to the easily accessible locations of both the support strap 22 and the counterforce strap 18, for example. Furthermore, the user may also apply a cold therapy device to her shoulder while using the shoulder brace 10 of the present disclosure.

In addition, it will be understood that the shoulder brace 10 and related methods greatly improve the ease of application of the shoulder brace 10 at the end of surgery. For example, the base layer 12 of the shoulder brace 10 is able to rest behind the user patient, who typically is in a seated or partially reclined position or in a side-lying position with the involved arm up. At the end of surgery, the base layer 12 of the shoulder brace 10 may be easily wrapped around a front area of the user patient, the side support attached to the base layer 12, the patient's arm placed in the sling, the sling attached to the side support, the sling strap 17 passed around the user's neck, and the counterforce strap 18 connected to the sling strap 17, as explained more above. The cold therapy device 51 may then easily be attached to the sling strap 17, as also described above. All of this can be applied without lifting the patient up or forward to pass straps around them, as is necessary with current brace designs, for example.

In addition, the cold therapy device 51 and related method describe above include several advantages. For example, the cold therapy device 51 has a comfortable contoured fit without additional straps typically wrapped around the patient's torso and under the opposite arm as well as wrapping around the upper involved arm and often under the armpit on the involved side. These additional straps in many conventional cold therapy devices add significantly to discomfort and complexity in applying and removing the cold therapy device used with any shoulder brace, for example, and may lead to post-operative swelling of the arm. At least because such additional straps are omitted in the cold therapy device 51 of the present disclosure, the cold therapy device 51 of the present disclosure minimizes post-operative swelling of the arm and related complications as well as providing greater comfort and ease of use for the patient.

The figures and description provided herein depict and describe preferred embodiments of a shoulder brace, a cold therapy device assembly for use with the shoulder brace, and related methods for purposes of illustration only. One skilled in the art will readily recognize from the foregoing discussion that alternative embodiments of the components illustrated herein may be employed without departing from the principles described herein. Thus, upon reading this disclosure, those of skill in the art will appreciate still additional alternative structural and functional designs. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the methods and components disclosed herein without departing from the spirit and scope defined in the appended claims.

1. A shoulder brace comprising:
a base layer having a first side and a second side;
a side support coupled to the first side;
a sling attached to the side support and having a sling strap; and
a counterforce strap having a first end coupled to the second side of the base layer and a second end attached to a portion of the sling strap, the counterforce strap applying a force to the sling strap in a direction toward the second side of the base layer.

2. The shoulder brace of claim 1, further comprising a hand grip attached to only one side of the sling.

3. The shoulder brace of claim 1, wherein the base layer is configured to be disposed around a waist of a user, the base layer further comprising additional padding for lumbar support disposed on the first side.

4. The shoulder brace of claim 1, wherein one or more of: (1) the side support is coupled to the first side of the base layer by a hook and loop mechanism; and (2) the side support is adjustably coupled to the base layer to accommodate various placements of the sling.

5. The shoulder brace of claim 1, wherein the first side of the base layer is disposed opposite to the second side of the base layer.

6. The shoulder brace of claim 1, wherein the sling strap includes a first end coupled to a first portion of the sling, a second end coupled to the second portion of the sling, and a middle portion disposed between the first and second ends and configured to extend across a shoulder of the user, and wherein the first portion of the sling is a front portion of the sling and the second portion of the sling is a rear portion of the sling.

7. The shoulder brace of claim 6, wherein the second end of the counterforce strap is coupled to the middle portion of the sling strap, the middle portion configured to be disposed in part on a chest of the user.

8. The shoulder brace of claim 1, wherein the sling strap further includes a ring, and the second end of the counterforce strap attaches to the ring to relieve weight of the sling strap on a neck of the user.

9. The shoulder brace of claim 1, wherein the sling includes a body having a front portion, a rear portion, an inside surface extending from the front portion to the rear portion, and a hand grip attached to the inside surface on one side of the body near the front portion, allowing a hand of a user to be easily inserted into the body of the sling to contact the hand grip.

10. The shoulder brace of claim 1, further comprising a support strap having a first end attached to the second side of the base layer and a second end removably attached to the side support disposed at the first side of the base layer, opposite to the second side of the base layer, to support the base layer.

11. The shoulder brace of claim 1, further comprising a cold therapy device assembly attached to the sling strap, wherein the cold therapy device assembly includes a cold therapy device having a first portion and a second portion, at least one connecting member coupled to the first portion of the cold therapy device, and at least one connecting member coupled to the second portion of the cold therapy device.

12. A method of supporting an arm of a user, the method comprising:
providing a base layer of a shoulder brace, the base layer configured to be disposed around a waist of a user;
coupling a side support to a first side of the base layer;
attaching a sling to the side support, the sling for receiving an arm of the user and having a sling strap configured to be disposed over a shoulder of the user to support the weight of the sling;
securing a first end of a counterforce strap to a second side of the base layer; and
securing a second end of the counterforce strap to a portion of the sling strap, applying a force to the sling strap in a direction toward the second side of the base layer.

13. The method of claim 12, wherein securing a second end of the counterforce strap to a portion of the sling strap comprises securing a second end of the counterforce strap to one or more of: (1) a portion of the sling strap configured to be disposed on a front area of the user; (2) a middle portion of the sling strap; and (3) a ring disposed on the sling strap.

14. The method of claim 12, further comprising attaching a first end of a support strap to the second side of the base layer and removably attaching a second end of the support strap to the side support disposed at the first side of the base layer, opposite to the second side of the base layer, to support the base layer.

15. The method of claim 12, further comprising coupling a first end of the sling strap to a first portion of the sling configured to be disposed near a front area of the user.

16. The method of claim 12, further comprising disposing additional padding for lumbar support on or in the base layer.

17. The method of claim 12, further comprising attaching a hand grip to an inside surface on one side of a body of the sling, allowing a hand of a user to be easily inserted into the body of the sling to contact the hand grip.

18. A cold therapy device assembly for use with a shoulder brace, the cold therapy device assembly comprising:
a cold therapy device having a first portion and a second portion,
at least one connecting member coupled to the first portion of the cold therapy device, and
at least one connecting member coupled to the second portion of the cold therapy device,
wherein the at least one connecting member coupled to the first portion of the cold therapy device is configured to be attached to a portion of a sling or a shoulder brace, and the at least one connecting member coupled to the second portion of the cold therapy device is configured to be attached to another portion of the sling or the shoulder brace.

19. The cold therapy device assembly of claim 18, wherein the at least one connecting member coupled to the first portion of the cold therapy device is at least one connecting strap and the at least one connecting member coupled to the second portion of the cold therapy device is at least one connecting strap, wherein each of the at least one connecting straps includes a set of connecting straps.

20. The cold therapy device assembly of claim 19, wherein the cold therapy device assembly comprises a first set of connecting straps having at least one connecting strap configured to be attached to a front portion of a sling strap of the shoulder brace and a second set of connecting straps having at least one connecting strap configured to be attached to a rear portion of the sling strap of the shoulder brace.

21. The cold therapy device assembly of claim 19, wherein the at least one connecting member coupled to the first portion of the cold therapy device is a first set of connecting straps having a first connecting strap and a second connecting strap, the first connecting strap having a first portion attached to a front portion of the cold therapy device, and a second portion configured to be disposed through a ring in the front portion of the sling strap, and the second connecting strap having a first portion coupled to the front portion of the cold therapy device and a second portion attached to a side portion of the cold therapy device, the side portion disposed on an upper arm area of a user.

22. The cold therapy device assembly of claim 19, wherein the at least one connecting member coupled to the second portion of the cold therapy device comprises a second set of connecting straps having a first connecting strap and a second connecting strap, the first connecting strap disposed over a rear portion of the sling strap and onto a rear portion of the cold therapy device, and the second connecting strap having a first portion attached to the rear portion of the cold therapy device and a second portion attached to a side portion of the cold therapy device.

23. The cold therapy device assembly of claim 18, wherein the cold therapy device includes one of a gel pad, an ice bag, a contoured device that circulates water to maintain consistent cryotherapy, or another pad that contours to the shoulder.

24. A method of applying a cold therapy device to a shoulder using one of a sling or a shoulder brace, the method comprising:
providing a cold therapy device adapted to be disposed on a shoulder of a user;
attaching a first portion of the cold therapy device to a portion of a sling or a shoulder brace by at least one connecting member; and
attaching a second portion of the cold therapy device to another portion of the sling or the shoulder brace by at least one other connecting member.

25. The method of claim 24, wherein attaching a first portion of the cold therapy device to a portion of a sling or a shoulder brace by at least one connecting member comprises attaching the first portion of the cold therapy device to a front portion of a sling strap of the sling by a first set of connecting straps including a first connecting strap and a second connecting strap, attaching the first connecting strap over the sling strap and onto the first portion of the cold therapy device and disposing the second connecting strap through a ring disposed on the sling strap.

26. The method of claim 24, wherein attaching a second portion of the cold therapy device to another portion of the sling or the shoulder brace by at least one connecting member comprises attaching the second portion of the cold therapy device to a rear portion of the sling strap by a second set of connecting straps including a first connecting strap and a second connecting strap, attaching the first connecting strap over the rear portion of the sling strap and onto the second portion of the cold therapy device and the second connecting strap over the rear portion of the sling strap and onto a side portion of the cold therapy device.

27. The method of claim 24, further comprising disposing a first portion of a cold therapy device under a front portion of a sling strap of a sling of a shoulder brace, and disposing a second portion of a cold therapy device under a rear portion of the sling strap of the sling of the shoulder brace.

28. The method of claim 24, further comprising securing a counterforce strap of the shoulder brace to a portion of the sling strap, applying a force to the sling strap in a direction away from the at least one connecting member attached to the front portion of the sling strap.
